(19) United States
(12) Patent Application Publication
(10) Pub. No.: US 2023/0285749 A1
(43) Pub. Date: Sep. 14, 2023

(54) ELECTRICALLY STIMULABLE CERVICAL VERTEBRAL SPINE IMPLANTS AND METHODS OF USE

(71) Applicant: Kutz Neurological Consultations, PLLC, Fricso, TX (US)

(72) Inventor: Scott C. Kutz, Frisco, TX (US)

(21) Appl. No.: 17/694,087

(22) Filed: Mar. 14, 2022

(51) Int. Cl. A61N 1/05; A61N 1/36; A61N 1/375

(52) U.S. Cl. CPC A61N 1/0558; A61N 1/36062; A61N 1/37518

(57) ABSTRACT

Electrically stimulable cervical vertebral spine implants and methods of use are disclosed herein. An example apparatus can include a curved body that is configured to conform to a portion of a cervical vertebra, an anchor member that extends away from the curved body, the anchor member having an aperture, and a securement member used to couple the anchor member and the curved body to the cervical vertebra in such a way that the curved body is oriented towards a spinal cord.

### CROSS-REFERENCE TO RELATED APPLICATION(S)

N/A.

### TECHNICAL FIELD

The present disclosure pertains to spinal implants and systems, and more particularly, but not by way of limitation, to electrically stimulable cervical vertebral spine implants and methods of use.

### SUMMARY

According to some embodiments, the present disclosure is directed to an apparatus comprising a curved body that is configured to conform to a portion of a cervical vertebra; an anchor member that extends away from the curved body, the anchor member comprising an aperture; and a securement member used to couple the anchor member and the curved body to the cervical vertebra in such a way that the curved body is oriented towards a spinal cord.

In one embodiment, an electrical conductor is associated with the curved body. A stimulator device can be included for delivering a current to the electrical conductor, wherein the electrical conductor is coupled to the stimulator device with wires.

In some embodiments the anchor member is oriented horizontally. In some embodiments the anchor member is oriented vertically. In one embodiment the anchor member contacts a posterior tubercle and/or posterior arches of the cervical vertebra.

In one embodiment the securement member is looped through the aperture of the anchor member and around the posterior tubercle and/or the posterior arches of the cervical vertebra. In one embodiment the securement member is a cinchable tie or a surgical suture.

According to some embodiments, the present disclosure is directed to an apparatus comprising a curved body that is configured to conform to a posterior portion of a cervical vertebra; an anchor member that extends away from the curved body, the anchor member comprising an aperture; a securement member used to couple the anchor member and the curved body to the posterior portion of the cervical vertebra in such a way that the curved body is oriented towards a spinal cord; an electrical conductor associated with the curved body; and a stimulator device for delivering a current to the electrical conductor, wherein the electrical conductor is coupled to the stimulator device with wires.

In some embodiments the anchor member is oriented horizontally or vertically. The anchor member can contact posterior arches of the cervical vertebra. In some embodiments, the securement member is looped through the aperture of the anchor member and around a posterior tubercle and/or the posterior arches of the cervical vertebra. The securement member can be a cinchable tie or a surgical suture.

According to some embodiments, the present disclosure is directed to a method comprising exposing a cervical vertebra of a patient using a posterior approach; inserting an implant into a spinal canal of the cervical vertebra in such a way that an electrical conductor of the implant faces a spinal cord of the patient; securing the implant to the cervical vertebra; coupling the electrical conductor to a stimulator device; and stimulating the spinal cord with current through the electrical conductor using the stimulator device.

In one embodiment securing the implant to the cervical vertebra includes looping a securement member through an aperture of the implant and around a posterior portion of the cervical vertebra. In one embodiment securing the implant to the cervical vertebra includes suturing the implant to the cervical vertebra. In another embodiment, securing the implant to the cervical vertebra includes suturing the implant to an adjacent cervical vertebra.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 is a perspective view of an example apparatus of the present disclosure installed on a cervical vertebra of a spine of a patient.

vertebra.

FIG. 2 is a top down view of an example implant installed on a cervical

FIGS. 3A and 3B collectively illustrate a portion of an example implant of the present disclosure.

FIGS. 3C and 3D collectively illustrate a portion of another example implant of the present disclosure.

FIG. 4 is a flowchart of an example method of installing and using an apparatus of the present disclosure.

FIG. 5 is a schematic view of an example computer system that can be used in accordance with the present disclosure.

### DETAILED DESCRIPTION

### Overview

The present disclosure is directed to electrically stimulable cervical vertebral spine implants and methods of use. Generally speaking, an example implant can be configured for placement on a cervical vertebral body. The implant can be, in some embodiments, secured to a posterior portion of the cervical vertebra. That is, during a procedure using a posterior approach to the spine, the implant can be configured for placement on a posterior structure of the cervical vertebra, such as the posterior tubercle and/or posterior arches. In other configurations or use cases, such as during a procedure using an anterior approach to the spine, the implant can be configured for placement on an anterior structure of the cervical vertebra. To be sure, an anterior approach to the cervical vertebral body is common with cervical fusion procedures. An implant of the present disclosure can be installed on the anterior portion of a cervical vertebral body or against a fusion device that spans two or more cervical vertebral bodies.

In one example implantation, the implant can be inserted into the spinal canal and contact an of the cervical vertebra near or over where the posterior arches connect at the posterior tubercle. In another example implantation, the implant can be attached to an outer surface posterior arches connect at the posterior tubercle. The exact placement of the implant can depend, at least in part on the type of vertebral body that the implant is being associated with. For example, a first placement configuration for an implant may be selected when the vertebral body is C1, a second placement configuration for an implant may be selected when the vertebral body is C2, and so forth.

In some embodiments, an implant disclosed herein can include a substantially flat body and an anchor member that extends substantially orthogonally to the flat body. One or more securement elements can be used to couple the implant to the cervical vertebra and/or an adjacent cervical vertebra. A securement element can loop around a structure of a cervical vertebral body and also through the anchor member to secure the implant in place.

In some embodiments, the implant can have an electrode/conductor associated therewith. The electrode/conductor can be associated with a stimulator device. The stimulator device can include a neurostimulator. In one embodiment, the implant and stimulator device can be electrically and communicatively coupled via a wired connection. In another example, the implant and stimulator device can be coupled wirelessly through an inductive means. In yet other embodiments, the stimulator device can be integrated with the implant, such as a system on a chip (SOC). Thus, some or all of the components of a traditional stimulator device can be integrated into the implant allowing the implant to operate autonomously or through remote control such as an application executing on a mobile device.

### Example Embodiments

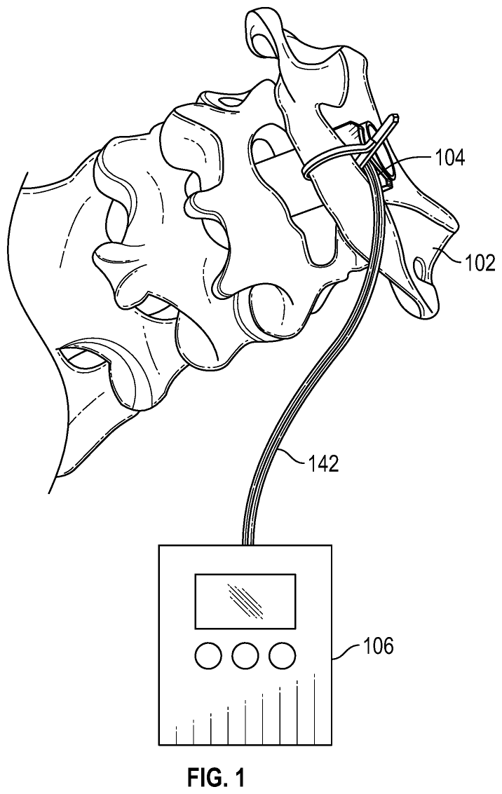

FIG. 1 is a perspective view of an environment where aspects of the present disclosure can be practiced. The environment can include a cervical vertebra 102, an implant 104, a stimulator device 106. For purposes of clarity and brevity, the examples provided herein will refer to C1 as the cervical vertebra 102. As noted, the implant 104 can be installed on any vertebra, and further onto any cervical vertebra.

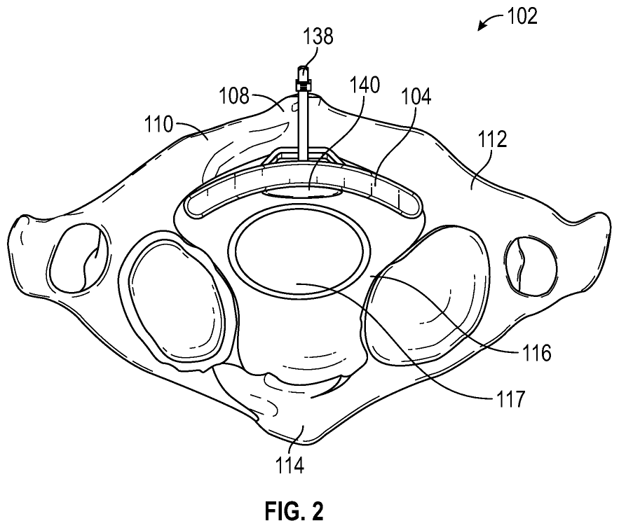

With reference to FIG. 2, the cervical vertebra 102 includes a posterior tubercle 108 and posterior arches 110 and 112. The cervical vertebra 102 also comprises an anterior tubercle 114 (in other vertebra this may include a spinous process) that is formed at the point where the posterior arches 110 and 112 converge. The posterior tubercle 108 and posterior arches 110 and 112 (along with lateral masses, anterior arches and anterior tubercle) cooperate to define a spinal canal 116. A spinal cord 117 extends through the spinal canal 116. To be sure, the posterior tubercle 108 extends posteriorly and the anterior tubercle 114 projects anteriorly.

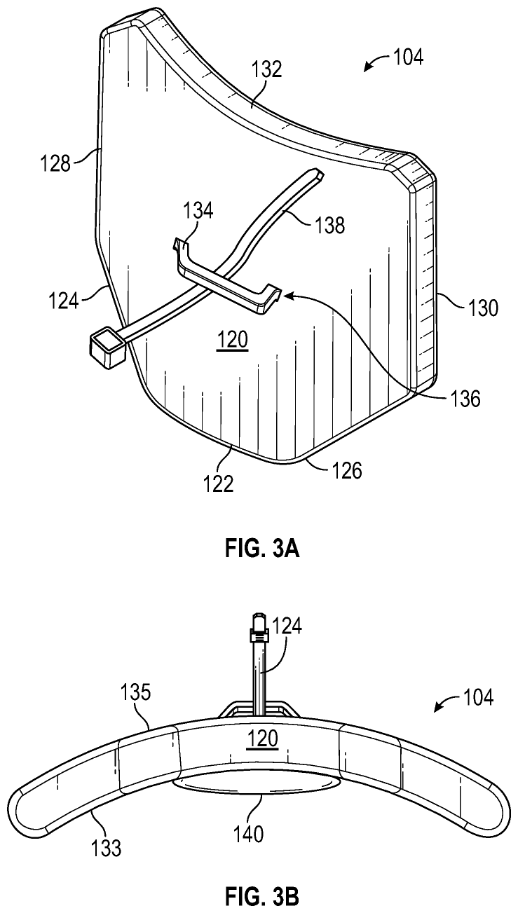

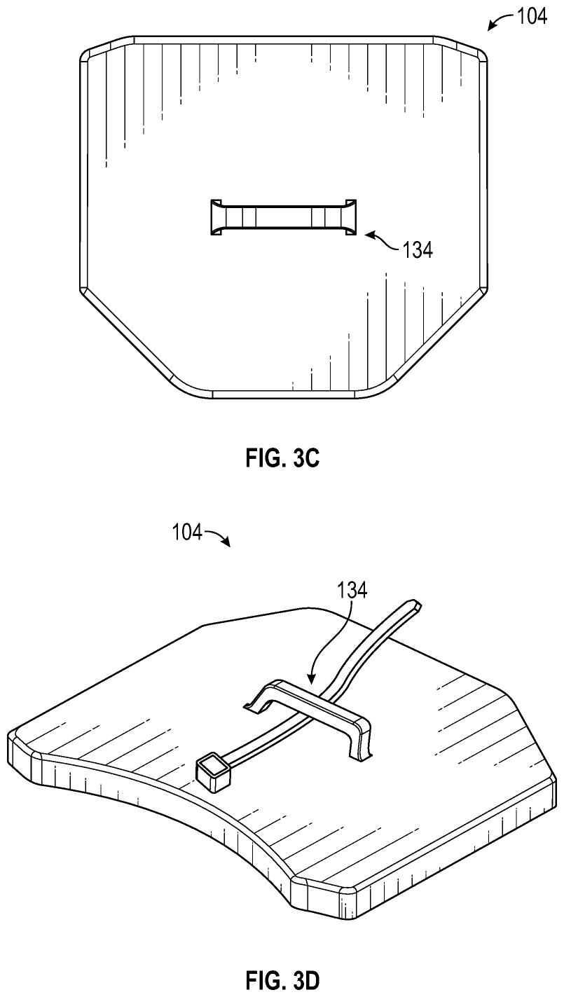

FIGS. 3A-B collectively illustrate in detail the implant 104. The implant 104 may comprise a body 120 having a generally polygonal shape. The body 120 can comprise a bottom edge 122 that includes two lateral tapered edges 124 and 126 that transition to two side edges 128 and 130. The body 120 also comprises a top edge 132. In some embodiments, the body 120 has a curved shape that conforms to an inside the posterior tubercle 108. That is, the implant 104 can conform to an arcuate shape of the anterior portion of the cervical vertebra 102. The body 120 includes a first surface 133 and a second surface 135.

The various components of the implant can be manufactured from a suitable biocompatible plastic or polymeric material. In one example, the body of the implant can be manufactured from polyetheretherketone (PEEK) and polyethylene, however other materials such as ceramics and composite materials can be used as well. In some embodiments, the material selected is a dielectric material that is unaffected by the electrical current emitted through an electrical conductor of the implant (as described in greater detail infra).

In some embodiments, the implant 104 comprises an anchor member 134 that extends away from the body 120. In some instances, the anchor member 134 extends orthogonally from the body 120. In other instances, the anchor member 134 extends from the body 120 at an angle other than orthogonal. The anchor member 134 can include an aperture 136 that receives a securement element 138 (also see FIG. 2). Rather than having an aperture, a suture could be threaded through the body of the anchor member 134 and looped around a portion of the cervical vertebra.

It will be understood that the anchor member 134 can extend away from the body 120 at a distance or length that allows the implant 104 to be secured to the cervical vertebra 102 but safely positioned away from the spinal cord 117. That is, it would be preferred to keep the implant 104 at a safe distance away from the spinal cord 117 to keep the implant from contacting the spinal cord 117. It will be noted that in FIGS. 3A and 3B the anchor member 134 is vertically oriented.

In some embodiments, the securement element 138 can include a cinchable tie. The securement element 138 can be inserted through the aperture 136 of the anchor member 134 and looped around the posterior tubercle 108. The securement element 138 can be cinched or otherwise secured to lock the implant in place against the posterior tubercle 108. In some embodiments, the securement element 138 is a suturing thread that is tied off by a surgeon.

While some embodiments contemplate securing the implant to the cervical vertebra that the implant is disposed within, in some embodiments, the implant can also be secured to an adjacent vertebra. For example, another securement element can be used to loop around a portion of C2 (such as a spinous process) as an additional anchor for the implant.

When installed on the cervical vertebra 102, the first surface 133 of the body 120 of the implant 104 faces an inner surface where the posterior arches 110 and 112 meet. The second surface 135 therefore faces towards the spinal cord 117. The anchor member 134 may contact the portion of the cervical vertebra 102 where the posterior arches 110 and 112 meet and define the posterior tubercle 108.

Referring briefly to FIGS. 3C and 3D, which illustrate another example configuration of the implant, with the exception that the anchor member 134 is horizontally oriented. It will be understood that the anchor member 134 can have an orientation that is between the vertical orientation of FIGS. 3A and 3B and the horizontal orientation of FIGS. 3C and 3D.

An electrical conductor 140 can be placed onto the second surface 135. The electrical conductor 140 can be coupled to the second surface 135 of the implant using, for example, an adhesive or fastener that would be known to one of ordinary skill in the art.

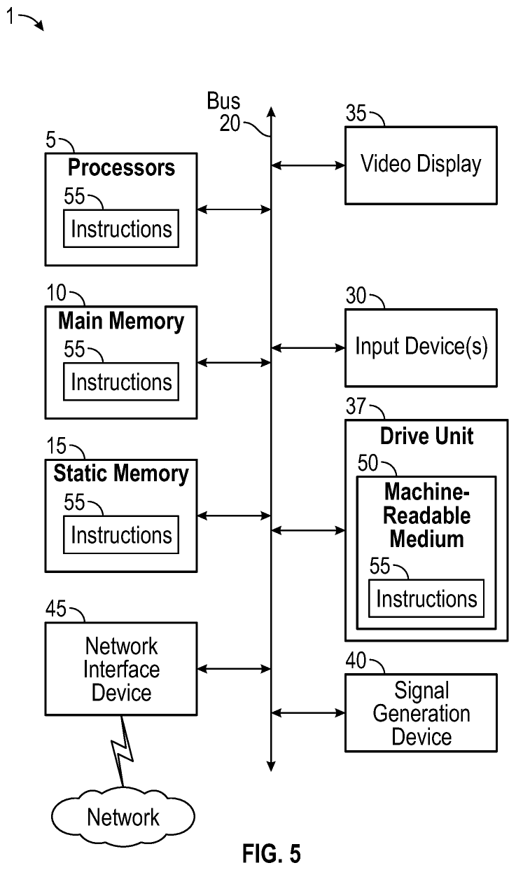

The electrical conductor 140 can be connected through wires 142 (see FIG. 1) to the stimulator device 106. The stimulator device 106 can be activated and provide a stimulating current to the electrical conductor 140 through the wire 142. In some embodiments, the stimulating current is adjusted to provide a desired effect to the spinal cord 117. The magnitude of the current can be selected based on the desired depth of penetration. For example, the magnitude of the current may vary depending on whether stimulation should penetrate into the white or gray matter of the spinal cord. A different magnitude of the current may be used when stimulating only the Pia matter or Arachnoid. Additional example structure of the stimulator device 106 can be found in FIG. 5. In general, the stimulator device 106 can be a dedicated device that is configured specifically to deliver the current. In other examples, the stimulator device 106 could include a Smartphone or other mobile device.

While some examples include wires, the implant 104 can include an inductive electrical element that can receive an inductive electrical current from the stimulator device 106. The inductive electrical element can be embedded into, or mounted on the body 120 of the implant. The inductive element can be activated and energized by a corresponding inductive electrical element in the stimulator device 106. In use, a user can place the inductive electrical element in the stimulator device 106 in proximity to the inductive element in the implant and deliver to activate the inductive element in the implant and deliver a current to the spinal cord.

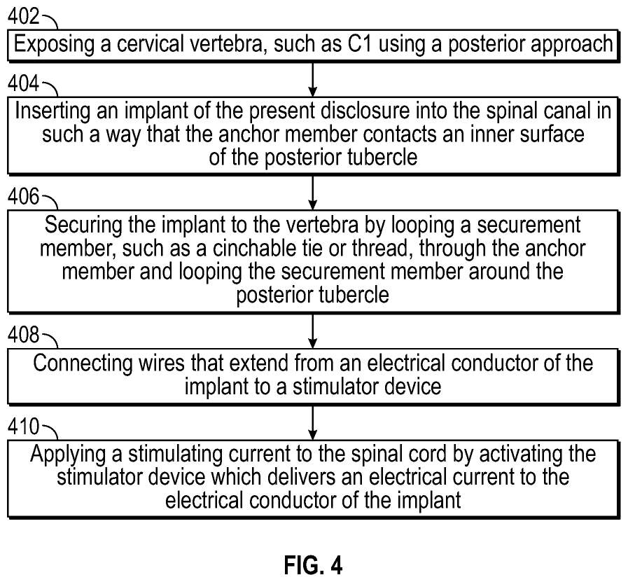

FIG. 4 is a flowchart of an example method for placing an implant of the present disclosure. The method includes a step 402 of exposing a cervical vertebra, such as C1. As noted above, this can include a posterior approach to the spine. When C1 is exposed, the method can include a step 404 of a surgeon inserting an implant of the present disclosure into the spinal canal in such a way that the anchor member contacts an inner surface of the posterior tubercle. Again, the inner surface of the posterior tubercle is the anterior surface of the posterior tubercle that helps define the portion of the spinal canal formed by C1. As noted above, the implant is inserted in such a way that an electrical conductor associated with the implant is facing the spinal cord.

The method can include a step 406 of securing the implant to the vertebra by looping a securement member, such as a cinchable tie or thread, through the anchor member and looping the securement member around the posterior tubercle. The method can include a step 408 of connecting wires that extend from an electrical conductor of the implant to a stimulator device. The surgeon can close the surgical opening, allowing the wires to extend through the skin of the patient.

In some embodiments, the method includes a step 410 of applying a stimulating current to the spinal cord by activating the stimulator device which delivers an electrical current to the electrical conductor of the implant. In some embodiments, this can include delivering the electrical current at a steady rate. In other embodiments, the electrical current can be pulsed according to a specified schedule or pattern.

FIG. 5 is a diagrammatic representation of an example machine in the form of a computer system 1, within which a set of instructions for causing the machine to perform any one or more of the methodologies discussed herein may be executed. In various example embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be an Internet-of-Things (IoT) device or system, personal computer (PC), a tablet PC, a set-top box (STB), a personal digital assistant (PDA), a cellular telephone, a portable music player (e.g., a portable hard drive audio device such as a Moving Picture Experts Group Audio Layer 3 (MP3) player), a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 1 includes a processor or multiple processor(s) 5 (e.g., a central processing unit (CPU), a graphics processing unit (GPU), or both), and a main memory 10 and static memory 15, which communicate with each other via a bus 20. The computer system 1 may further include a video display 35 (e.g., a liquid crystal display (LCD)). The computer system 1 may also include an alpha-numeric input device(s) 30 (e.g., a keyboard), a cursor control device (e.g., a mouse), a voice recognition or biometric verification unit (not shown), a drive unit 37 (also referred to as disk drive unit), a signal generation device 40 (e.g., a speaker), and a network interface device 45. The computer system 1 may further include a data encryption module (not shown) to encrypt data.

The drive unit 37 includes a computer or machine-readable medium 50 on which is stored one or more sets of instructions and data structures (e.g., instructions 55) embodying or utilizing any one or more of the methodologies or functions described herein. The instructions 55 may also reside, completely or at least partially, within the main memory 10 and/or within the processor(s) 5 during execution thereof by the computer system 1. The main memory 10 and the processor(s) 5 may also constitute machine-readable media.

The instructions 55 may further be transmitted or received over a network via the network interface device 45 utilizing any one of a number of well-known transfer protocols (e.g., Hyper Text Transfer Protocol (HTTP)). While the machine-readable medium 50 is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read only memory (ROM), and the like. The example embodiments described herein may be implemented in an operating environment comprising software installed on a computer, in hardware, or in a combination of software and hardware.

One skilled in the art will recognize that the Internet service may be configured to provide Internet access to one or more computing devices that are coupled to the Internet service, and that the computing devices may include one or more processors, buses, memory devices, display devices, input/output devices, and the like. Furthermore, those skilled in the art may appreciate that the Internet service may be coupled to one or more databases, repositories, servers, and the like, which may be utilized in order to implement any of the embodiments of the disclosure as described herein.

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description, but is not intended to be exhaustive or limited to the present technology in the form disclosed. Many modifications and variations will be apparent to those of ordinary skill in the art without departing from the scope and spirit of the present technology. Exemplary embodiments were chosen and described in order to best explain the principles of the present technology and its practical application, and to enable others of ordinary skill in the art to understand the present technology for various embodiments with various modifications as are suited to the particular use contemplated.

If any disclosures are incorporated herein by reference and such incorporated disclosures conflict in part and/or in whole with the present disclosure, then to the extent of conflict, and/or broader disclosure, and/or broader definition of terms, the present disclosure controls. If such incorporated disclosures conflict in part and/or in whole with one another, then to the extent of conflict, the later-dated disclosure controls.

Aspects of the present technology are described above with reference to flowchart illustrations and/or block diagrams of methods, apparatus (systems) and computer program products according to embodiments of the present technology. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

Any and/or all elements, as disclosed herein, can be formed from a same, structurally continuous piece, such as being unitary, and/or be separately manufactured and/or connected, such as being an assembly and/or modules. Any and/or all elements, as disclosed herein, can be manufactured via any manufacturing processes, whether additive manufacturing, subtractive manufacturing and/or other any other types of manufacturing. For example, some manufacturing processes include three-dimensional (3D) printing, laser cutting, computer numerical control (CNC) routing, milling, pressing, stamping, vacuum forming, hydroforming, injection molding, lithography and/or others.

Any and/or all elements, as disclosed herein, can include, whether partially and/ or fully, a solid, including a metal, a mineral, a ceramic, an amorphous solid, such as glass, a glass-ceramic, an organic solid, such as and/or a polymer, such as rubber, a composite material, a semiconductor, a nano-material, a biomaterial and/or any combinations thereof. Any and/or all elements, as disclosed herein, can include, whether partially and/or fully, a coating, including an informational coating, such as ink, an adhesive coating, a melt-adhesive coating, such as vacuum seal and/or heat seal, a release coating, such as tape liner, a low surface energy coating, an optical coating, such as for tint, color, hue, saturation, tone, shade, transparency, translucency, non-transparency, luminescence, anti-reflection and/or holographic, a photo-sensitive coating, an electronic and/or thermal property coating, such as for passivity, insulation, resistance or conduction, a magnetic coating, a water-resistant and/or waterproof coating, a scented coating and/or any combinations thereof.

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description, but is not intended to be exhaustive or limited to the present technology in the form disclosed. Many modifications and variations will be apparent to those of ordinary skill in the art without departing from the scope and spirit of the present technology. Exemplary embodiments were chosen and described in order to best explain the principles of the present technology and its practical application, and to enable others of ordinary skill in the art to understand the present technology for various embodiments with various modifications as are suited to the particular use contemplated.

If any disclosures are incorporated herein by reference and such incorporated disclosures conflict in part and/or in whole with the present disclosure, then to the extent of conflict, and/or broader disclosure, and/or broader definition of terms, the present disclosure controls. If such incorporated disclosures conflict in part and/or in whole with one another, then to the extent of conflict, the later-dated disclosure controls.

The terminology used herein can imply direct or indirect, full or partial, temporary or permanent, immediate or delayed, synchronous or asynchronous, action or inaction. For example, when an element is referred to as being “on,” “connected” or “coupled” to another element, then the element can be directly on, connected or coupled to the other element and/or intervening elements may be present, including indirect and/or direct variants. In contrast, when an element is referred to as being “directly connected” or “directly coupled” to another element, there are no intervening elements present.

Although the terms first, second, etc. may be used herein to describe various elements, components, regions, layers and/or sections, these elements, components, regions, layers and/or sections should not necessarily be limited by such terms. These terms are only used to distinguish one element, component, region, layer or section from another element, component, region, layer or section. Thus, a first element, component, region, layer or section discussed below could be termed a second element, component, region, layer or section without departing from the teachings of the present disclosure.

The terminology used herein is for the purpose of describing particular embodiments only and is not intended to be necessarily limiting of the disclosure. As used herein, the singular forms “a,” “an” and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence or addition of one or more other features, integers, steps, operations, elements, components, and/or groups thereof.

Example embodiments of the present disclosure are described herein with reference to illustrations of idealized embodiments (and intermediate structures) of the present disclosure. As such, variations from the shapes of the illustrations as a result, for example, of manufacturing techniques and/or tolerances, are to be expected. Thus, the example embodiments of the present disclosure should not be construed as necessarily limited to the particular shapes of regions illustrated herein, but are to include deviations in shapes that result, for example, from manufacturing.

In this description, for purposes of explanation and not limitation, specific details are set forth, such as particular embodiments, procedures, techniques, etc. in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

That which is claimed is:

1. An apparatus comprising:
a curved body that is configured to conform to a portion of a cervical vertebra;
an anchor member that extends away from the curved body, the anchor member comprising an aperture; and
a securement member used to couple the anchor member and the curved body to the cervical vertebra in such a way that the curved body is oriented towards a spinal cord.

2. The apparatus according to claim 1, further comprising an electrical conductor associated with the curved body.

3. The apparatus according to claim 2, further comprising a stimulator device for delivering a current to the electrical conductor, wherein the electrical conductor is coupled to the stimulator device with wires.

4. The apparatus according to claim 1, wherein the anchor member is oriented horizontally.

5. The apparatus according to claim 1, wherein the anchor member is oriented vertically.

6. The apparatus according to claim 1, wherein the anchor member contacts a posterior tubercle and/or posterior arches of the cervical vertebra.

7. The apparatus according to claim 6, wherein the securement member is looped through the aperture of the anchor member and around the posterior tubercle and/or the posterior arches of the cervical vertebra.

8. The apparatus according to claim 7, wherein the securement member is a cinchable tie or a surgical suture.

9. An apparatus comprising:
a curved body that is configured to conform to a posterior portion of a cervical vertebra;
an anchor member that extends away from the curved body, the anchor member comprising an aperture;
a securement member used to couple the anchor member and the curved body to the posterior portion of the cervical vertebra in such a way that the curved body is oriented towards a spinal cord;
an electrical conductor associated with the curved body; and
a stimulator device for delivering a current to the electrical conductor, wherein the electrical conductor is coupled to the stimulator device with wires.

10. The apparatus according to claim 9, wherein the anchor member is oriented horizontally or vertically.

11. The apparatus according to claim 9, wherein the anchor member contacts posterior arches of the cervical vertebra.

12. The apparatus according to claim 11, wherein the securement member is looped through the aperture of the anchor member and around a posterior tubercle and/or the posterior arches of the cervical vertebra.

13. The apparatus according to claim 12, wherein the securement member is a cinchable tie or a surgical suture.

14. A method comprising:
exposing a cervical vertebra of a patient using a posterior approach;
inserting an implant into a spinal canal of the cervical vertebra in such a way that an electrical conductor of the implant faces a spinal cord of the patient;
securing the implant to the cervical vertebra;
coupling the electrical conductor to a stimulator device; and
stimulating the spinal cord with current through the electrical conductor using the stimulator device.

15. The method according to claim 14, wherein securing the implant to the cervical vertebra includes looping a securement member through an aperture of the implant and around a posterior portion of the cervical vertebra.

16. The method according to claim 14, wherein securing the implant to the cervical vertebra includes suturing the implant to the cervical vertebra.

17. The method according to claim 14, wherein securing the implant to the cervical vertebra includes suturing the implant to an adjacent cervical vertebra.
